(19) United States
(12) Patent Application Publication
(10) Pub. No.: US 2018/0280343 A1
(43) Pub. Date: Oct. 4, 2018

(54) TOPICAL SILIBININ FORMULATIONS AND USES THEREFOR

(71) Applicants: Theresa PACHECO, Denver, CO (US); Thomas ANCHORDOQUY, Lakewood, CO (US); Rajesh AGARWAL, Lakewood, CO (US); Neera TEWARI-SINGH, Aurora, CO (US)

(72) Inventors: Theresa PACHECO, Denver, CO (US); Thomas ANCHORDOQUY, Lakewood, CO (US); Rajesh AGARWAL, Lakewood, CO (US); Neera TEWARI-SINGH, Aurora, CO (US)

(21) Appl. No.: 15/765,340

(22) PCT Filed: Oct. 3, 2016
(86) PCT No.: PCT/US16/55201

§ 371 Date: Apr. 2, 2018

Related U.S. Application Data
(60) Provisional application No. 62/236,547, filed on Oct. 2, 2015.

(51) Int. Cl. A61K 31/357; A61K 47/10; A61K 47/14; A61K 47/22; A61K 47/26; A61K 47/34; A61K 47/46; A61K 9/00; A61P 17/02; A61P 17/04; A61P 17/16; A61K 8/27; A61K 8/28; A61K 8/34; A61K 8/37; A61K 8/44; A61K 8/49; A61Q 19/08

(52) U.S. Cl. CPC A61K 31/357; A61K 45/06; A61K 47/14; A61K 47/22; A61K 47/26; A61K 47/34; A61K 47/46; A61K 9/0014; A61P 17/02; A61P 17/04; A61P 17/16; A61K 8/27; A61K 8/28; A61K 8/345; A61K 8/375; A61K 8/445; A61K 8/4946; A61K 8/4966; A61Q 19/08; A61K 2300/00; A61K 47/10

(57) ABSTRACT

Topical compositions containing silibinin with enhanced silibinin penetration into the dermis. The compositions are useful for the treatment or prevention of skin damage or disorders.

### GOVERNMENT INTEREST

This invention was made with U.S. Government support under grant numbers RO1 CA140368 and U54ES-015678 awarded by the National Institutes of Health (NIH). The U.S. Government has certain rights in this invention.

### TECHNICAL FIELD

This disclosure describes compositions for topical administration to a subject. In particular, this disclosure relates to compositions containing silibinin having enhanced skin penetration. This disclosure further relates to methods of making and using the compositions in the prevention and treatment of a variety of skin conditions.

### BACKGROUND

Milk thistle belongs to the aster family (Asteraceae or Compositae) that includes daisies, thistles, and artichokes. Silymarin (CAS No. 65666-07-1), a polyphenolic flavonoid mixture, belonging to the group of the flavanolignans, can be isolated from milk thistle, Silybum marianum (L.) Gaertn. Silymarin is composed mainly of three isomers: silidianin, silichristin, and silibinin. The biological activities of silymarin are primarily associated with silibinin (Am. J. Health Syst. Pharm. 56: 1195-1197 (1999); Biochem. Pharmacol. 48: 753-759 (1994)). Silibinin (also referred to as silybin, silibirin, silibinine, silibinina, filybine or flavobin) ((CAS No. 22888-70-6); C₂₅H₂₂O₁₀; 2R,3R-3,5,7-trihydroxy-2[(2R,3R)-2-(4-hydroxy-3-methoxyphenyl)-3-(hydroxymethyl)-2,3-dihydro-1,4-benzodioxin-7-yl]-2,3-dihydrochromen-4-one)), exists as a mixture of two diastereomers, silibinin A and silibinin B.

Extracts of milk thistle containing silibinin have been used to treat various liver ailments for the last three decades. Silibinin is a strong antioxidant that prevents oxidant-induced lipid peroxidation by scavenging free radicals, thereby preventing or minimizing liver injury caused by many toxic compounds including ethanol, benzopyrene, carbon tetrachloride and bacterial endotoxins. Silibinin is also an effective anticancer and anti-angiogenic agent that has been shown to interact with a number of proliferative cell signaling pathways, inhibiting proliferation and stimulating apoptosis. In addition, silibinin has a wide therapeutic window, with doses in excess of 13 grams per day shown to be safe in human Phase I and Phase II clinical studies (Flaig, T. W., et al., Invest New Drugs, 25(2):139-46, 2007; Flaig, T. W., et al., Prostate, 70(8):848-55, 2010).

Animal experiments have shown that silibinin interacts with proliferative cell signaling pathways, inhibiting tyrosine kinase inhibitor (TKI)-related proliferation and stimulating apoptotic pathways. Silibinin has been found to possess a wide variety of biological activities, including hepato-protection (Saliou, C., et. al., FEBS Letters, 440:8-12, 1998) and inhibition of breast (Zi, X. L., et al., Clinical Cancer Research, 4:1055-1064, 1998), prostate (Zi, X. L., et al., Cancer Research, 58:1920-1929, 1998; and Singh, R. P., et al., Clin. Cancer Res., 14(23):7773-80, 2008), colon (Kaur, M., et al., Mol. Cancer Ther., 8(8):2366-74, 2009) and skin (Lahiri-Chatterjee, M., et al., Cancer Research 59:622-632, 1999) tumor development, growth and progression. Additionally, mice fed silibinin for 10 weeks showed inhibition of lung tumor growth and regression in established lung adenocarcinomas. In these studies, both tumor number and tumor size were decreased, along with microvessel size and antioangiogenic activity. (Tyagi, A., et al., Cancer Prev. Res., 2(1):74-83, 2009)

Previous investigations of the effects of silibinin and/or silymarin on the skin have demonstrated inhibition of photocarcinogenesis (Katiyar, S. K., et al., JNCI 89(8):556-566, 1997), inhibition of inflammation (Zi, X. L., et al., Mol Carcinogenesis, 26:321-333, 1999), reversal of skin aging (U.S. Pat. Nos. 4,749,573; 4,997,649; 4,895,839) and effective treatment of dermatological conditions, such as psoriasis and dermatitis (EP Patent No. 0552439).

Silibinin has also been proven to be an effective chemopreventive in mouse skin cancer models. For example, silymarin was shown to cause both tumor growth inhibition and regression of established skin papillomas in SENCAR mice (Singh, R. P., et al., Carcinogenesis, 23(3):499-510, 2002). Topical application of silibinin immediately prior to, or immediately after, UV radiation accelerated the removal of thymine-dimers in epidermis of SKH-1 mice, suggesting a mechanism other than, or in addition to, a sunscreen effect (Dhanalakshmi, S., et al., Carcinogenesis, 25(8):1459-65, 2004). Topical or dietary administration of silibinin immediately prior to, or immediately after UV radiation protected against photocarcinogenesis in SKH-1 hairless mice (Mallikarjuna, G., et al., Cancer Res, 64(17):6349-56, 2004). Silibinin applied topically or administered through dietary supplementation before, or immediately after radiation, inhibited UV-B induced mitogenic and survival signaling in SKH-1 mouse skin (Gu, M., et al., Carcinogenesis, 26(8):1404-13, 2005). Dietary silibinin in SKH-1 mice resulted in a significant decrease in markers associated with tumor proliferation, DNA damage and apoptotic sunburn cells in epidermis (Gu, M., et al., Cancer Epidemiol Biomarkers Prev, 14(5):1344-9, 2005). In SKH-1 mice chronically exposed to UVB irradiation silibinin differentially affects molecular markers associated with cell growth, signaling and apoptosis in skin and induced tumors (Gu, M., et al., Mol Cancer Ther., 5(8):2121-9, 2006). Both topical and dietary silibinin protected against photocarcinogenesis in SKH-1 mice exposed to UVB radiation, as measured by tumor multiplicity, tumor volume and delay in tumor formation (Gu, M., et al., Cancer Res, 67(7):3483-91, 2007).

Unfortunately, silibinin is not readily soluble in aqueous or lipophilic phases. Additionally, following absorption, silibinin is metabolized to glucuronide and sulfate conjugates and rapidly cleared in the urine. These chemical and pharmacokinetic properties result in poor bioavailability and a short half-life for silibinin, which limits its effectiveness following oral administration. This has led to a search for effective means to achieve elevated silibinin concentrations in the skin. Previous methods studied include converting silibinin into lipophilic complexes with phospholipids (U.S. Pat. No. 4,764,508) or chemical modification of silibinin to form more soluble salts, such as the 3,11-dihemisuccinate salt of silibinin (U.S. Pat. No. 5,196,448). Absorption into the skin is known to be dependent on molecular size, and thus complexation limits absorption because it increases the molecular size that must penetrate into the epidermis.

Thus, there is a need for more effective topical silibinin formulations in order to realize the effective use of silibinin treating skin diseases, disorders, and injuries. To this end, formulations are needed that, (i) successfully deliver effective concentrations of silibinin into the epidermis and dermis, (ii) stabilize the active form of silibinin, and (iii) maintain silibinin activity and skin penetration properties in the presence of a wide range of excipients that may enhance silibinin therapeutic effect and/or add provide additional benefits, such as sunscreens.

### SUMMARY

This disclosure provides compositions of the antioxidant, anti-inflammatory, anticancer and antiangiogenic agent, silibinin in topical formulations that enhance the penetration of silibinin into the dermis.

The topical silibinin formulations of this disclosure include formulation components that enhance the penetration of silibinin into the dermis while simultaneously permitting the solubilization of therapeutically sufficient concentrations of the notoriously insoluble silibinin, while maintaining the stability of the overall formulation. For example, pH adjusting agents may be included in the topical formulations of this disclosure. Broad spectrum UV (UVA and/or UVB) sunscreens may be included in the topical compositions of this disclosure. These topical formulations may also include one or more moisturizers, stability enhancing agents, emulsifiers, vitamins, surfactants, aloe plant components, fragrances, and preservatives.

These topical formulations of silibinin provide ultraviolet and ionizing radiation protection and reverse or slow the progression of skin disorders, including skin cancers. The formulations of this disclosure are also useful in the treatment of subjects that have suffered skin damage from solar or therapeutic radiation, organ transplant patients at high risk for skin cancers due to immunosuppression, or patients suffering from disorders such as ataxia-telangiectasia and xeroderma pigmentosum, which are associated with increased risk of skin cancer. Such treatment may include the partial or complete reversal of pre-malignant changes that are associated with the development of skin cancer. These topical formulations prevent and treat chemically-induced skin damage caused by cytotoxic and vesicant chemical warfare agents such as mustard gas, sulfur mustard, and nitrogen mustard. These topical formulations prevent and treat environmentally-induced skin damages such as UVA and UVB-induced skin damages such as sunburn/tanning/skin thickening. These topical formulations prevent and treat environmentally-induced skin damage such as radiodermatitis/fibrosis associated with radiotherapy.

The enhanced penetration of silibinin into layers of skin is achieved with the compositions of this disclosure with formulation vehicles that are shelf stable and tolerant to the addition of excipients and therapeutic agents, and do not require complexation of silibinin.

### BRIEF DESCRIPTION OF DRAWINGS

FIG. 1 shows decreased DNA damage in irradiated mouse skin cells exposed to several silibinin-containing compositions of this disclosure.

FIG. 2 shows surprisingly high penetration of silibinin from compositions of this disclosure into mouse skin compared with penetration from control formulations.

FIGS. 3A and 3B show the reduction in thymine dimer formation caused by UVB irradiation of mouse skin following application of topical silibinin compositions of this disclosure. These figures show thymine dimer staining (black in FIG. 3A) representing DNA damage is markedly reduced by topical silibinin (FIG. 3B).

FIG. 4 shows the in vitro skin penetration testing of five silibinin formulations of this disclosure containing additional sunscreen compound concentrations, as described in Example 4.

FIG. 5 is a time line depicting the in vivo sulfur mustard exposure of the SKH-1 hairless mouse dorsal skin and silibinin treatment protocol.

FIG. 6A shows microvesication (epidermal-dermal separation or microblistering) in the mouse skin samples collected in the in vivo testing protocol outlined in FIG. 5. Percentages in the quantitation of microvesication (FIG. 6B) indicate the amount of reduction in microvesication compared to SM control (black bar). Silibinin, whether in acetone or formulation improves protection over carrier formulation (FB) alone. Data presented are the mean±SEM. *, p<0.05 compared to SM exposed group. SM, sulfur mustard; SB/Ace, Silibinin (in acetone); SB-1% F, Silibinin formulation 1%; SB-2% F, Silibinin formulation 2%; FB, formulation base. Arrows, microvesication (epidermal-dermal separation).

FIG. 7A shows myeloperoxidase (MPO) levels in the mouse skin samples collected in the in vivo testing protocol outlined in FIG. 5. Percentages in the quantitation of fold change (FIG. 7B) indicate the amount of reduction in MPO compared to SM control (black bar). Silibinin, whether in acetone or formulation improves protection over carrier formulation (FB) alone. Data presented are the mean±SEM. Abbreviations the same as FIG. 6.

FIG. 8 shows cyclooxygenase-2 (COX-2) levels in the mouse skin samples collected in the in vivo testing protocol outlined in FIG. 5. Data presented are the mean±SEM. *, p<0.05 compared to SM exposed group. Abbreviations the same as FIG. 6.

FIG. 9A shows trichrome staining showing collagen changes in the mouse skin samples collected in the in vivo testing protocol outlined in FIG. 5. Collagen formation, a marker of healing, is enhanced by formulations containing silibinin. Only the formulations of this disclosure increased collagen formation. Percentages in the quantitation data (FIG. 9B) are amount of increased collagen staining in comparison to sulfur mustard (SM) control. * indicates significantly more collagen staining (P<0.05) Data presented are the mean±SEM. *, p<0.05 compared to SM exposed group n=4-8). Abbreviations the same as FIG. 6.

FIGS. 10A and 10B demonstrate that silibinin treatment prevents and reverses 2 chloroethyl ethyl sulfide ( ) (CEES)-induced decreases in the viability of skin epidermal cells and fibroblasts by MTT assay. FIG. 10A shows the effect on cell viability in mouse epidermal JB6 cells, and FIG. 10B shows the effect in primary skin fibroblasts. Data presented are mean±SEM (n=3-5). *, p<0.05 compared to 0.35 mM CEES treated group. Control, vehicle (DMSO) control; SB, silibinin. CEES treatment: 24 h.

FIGS. 11A, and 11B demonstrate that silibinin treatment prevents and reverses CEES-induced decreases in DNA synthesis in the skin epidermal cells and fibroblasts by BrdU assay. FIG. 11A shows the effect on DNA synthesis in mouse epidermal JB6 cells, and FIG. 11B shows the effect in primary skin fibroblasts. Data presented are mean±SEM (n=3-4). *, p<0.05 compared to 0.5 mM CEES treated group. Control, vehicle (DMSO) control; SB, silibinin. CEES treatment: 24 h.

FIGS. 12A, 12B, and 12C demonstrate that silibinin treatment prevents and reverses CEES-induced DNA damage in the skin epidermal cells and fibroblasts by a comet assay. FIG. 12A shows photomicrographs of primary skin fibroblasts following indicated drug, formulation, or vehicle treatment. FIG. 12B shows the tail extent moment in mouse epidermal JB6 cells following the specified treatments. FIG. 12C shows the tail extent moment in primary skin fibroblasts following the specified treatments. Data presented are mean±SEM (n=3). *, p<0.05 compared to 0.5 mM CEES treated group. UC, untreated control; VC, vehicle (DMSO) control; SB, silibinin. CEES treatment: 1.5 h.

FIGS. 13A, 13B, and 13C demonstrate that silibinin treatment prevents and reverses CEES-induced DNA damage in skin epidermal cells. FIG. 13A shows the H2A.X ser139, and p53 ser15 phosphorylations in mouse epidermal cells at the indicated times and treatments. FIG. 13B shows the integrated density of the gel staining in for H2A.X ser139 phosphorylation at the indicated times and treatments. FIG. 13C shows the integrated density of the gel staining in for p53 ser15 phosphorylation at the indicated times and treatments. untreated control; VC, vehicle (acetone) control. CEES treatment; CEES treatment; 4 h.

### DESCRIPTION OF EMBODIMENTS

The present invention provides compositions and methods for the topical administration of a therapeutically-effective amount of silibinin to layers within the skin in order to improve the condition of the skin. Accordingly, the present disclosure provides compositions adapted for topical administration of silibinin, and methods for using such topical compositions containing silibinin. Methods provided by this disclosure include the topical delivery of silibinin in a stable form for local delivery to produce therapeutically effective levels of silibinin to the dermis and epidermis, wherein the silibinin dose delivered to the skin has therapeutic effects.

The term “physiologically acceptable” substance means that the substance would cause no significant adverse health effect when administered to the subject. Preferably, a “physiologically acceptable” substance would cause little or no allergic response and would be suitable for use in contact with human skin without undue toxicity, incompatibility and instability.

“Photodermatosis” are disorders induced by UV radiation. Mediated (toxic or allergic reactions to sunlight) and idiopathic (urticaria solaris, polymorphic dermatitis, actinic reticuloid) photodermatosis, as well as those liable to be worsened by sunlight (pellagra, lupus erythematosus, pemphigus erythematosus, xeroderma pigmentosum, dermatomyositis) may be considered as somewhat rare consequences of exposure to light. Conversely, direct photodermatoses are by far more frequent. Photodermatosis may appear as erythema/edema vesiculosum reaction (acute damage), photo-aging and photo-carcinogenesis (chronic damage).

“Radiation dermatitis” is a skin disease associated with prolonged exposure to radiation. This includes patients undergoing radiation therapy (in North America), or radiotherapy (in the UK and Australia) also called radiation oncology, and sometimes abbreviated “XRT,” as a medical use of ionizing radiation, typically as part of cancer treatment to control malignant cells.

### Topical Compositions

Milk thistle belongs to the aster family (Asteraceae or Compositae) that includes daisies, thistles, and artichokes. Silymarin (CAS No. 65666-07-1), a polyphenolic flavonoid mixture, belonging to the group of the flavanolignans, can be isolated from milk thistle, Silybum marianum (L.) Gaertn. Silymarin is composed mainly of three isomers: silidianin, silichristin, and the major component, silibinin. The biological activities of silymarin are primarily associated with silibinin (Am. J. Health Syst. Pharm. 56: 1195-1197 (1999); Biochem. Pharmacol. 48: 753-759 (1994)). Silibinin (CAS No. 22888-70-6), also known as “silybin,” exists as a mixture of two diastereomers, silibinin A and silibinin B, that are present in silymarin extracts at approximately a 1:1 ratio. The term “silibinin,” as used throughout this disclosure, encompasses silibinin A alone, silibinin B alone, as well as mixtures thereof, including a 1:1 mixture of silibinin A and silibinin B. Silibinin can be purchased commercially, for example from SIGMA™ (Catalog No. S0417). Thus, topical formulations of this disclosure may be formulated to contain predominately the silibinin diastereomer A, or predominately the silibinin diastereomer B, or a mixture of silibinin diastereomers A and B in a ratio of approximately 1:1.

In addition to the silibinin content, the topical compositions of this disclosure may also be substantially free, or completely free of other lignan compounds. As used herein, the term “substantially free” means a composition containing less than 0.5% of the stated ingredient, which ingredient may be regarded as a contaminant. As used herein, the term “completely free” means a composition containing undetectable amounts of the stated ingredient, which ingredient may also be regarded as a contaminant.

For example, the topical compositions of this disclosure may be substantially free, or completely free, of one or more lignans selected from silydianin, silychristin isosilybin, sauriol, licarin, saucernetin, saucerneol, niranthin, Phyllanthin, manassantins, matairesinol, hydroxymatairesinol, oxomatairesinol, saminol, americanin, arctiin, arctigenin, lariciresinol, isolariciresinol, secoisolariciresinol, secoisolariciresinol diglycoside, rubrisandrin, egonol, masutakeside, styraxlignolide, lappaol, diarctigenin, interiotherin, schisandrol, schisandrin, sesamin, sesaminol, episesamin, episesaminol, sesamolin, verbascoside, tetrahydrocurcumin, rosmarinic acid, chlorogenic acid, guaiaretic acid, dihydroguiaretic acid, nor-dihydroguiaretic acid, alpha-conidendrin, liovil, picearesinol, syringaresinol, and nortrachelogenin.

Previous formulations and studies have used silymarin extracts. But the use of extracts in these studies creates a number of problems. For example, the composition of the extract usually is not completely determined, resulting in uncertainty in the relationship of various chemical components and their biological activities. Furthermore, unidentified components present in extracts can cause adverse effects. Thus, the topical compositions of this disclosure may be substantially free or completely free of lignans present in silymarin extracts other than silibinin.

The topical compositions of the present invention contain silibinin in a topical vehicle that can enhance the penetration of the silibinin into the dermal layers of the skin. Effectiveness of the topical compositions of this disclosure requires percutaneous absorption and bioavailability of silibinin to the dermis. Thus, the compositions and methods of this disclosure require silibinin penetration through the stratum corneum into the epidermal and dermal layers of the skin or scalp, or penetration into the surface of the hair or nails, as well as sufficient distribution to the sites targeted for pharmacologic action.

Preferred compositions of this disclosure include silibinin in concentrations between 0.001% and 15%, preferentially at concentrations between 0.1% and 10%, and more preferentially at concentrations between 0.2% and 5%, by weight, based on the composition. Preferably, silibinin is present in the composition at a concentration of from about 1% to about 5% by weight. More preferably, it is present in the composition at a concentration of about 2.0%, by weight.

To facilitate percutaneous absorption of silibinin, the topical compositions of this disclosure may include chemicals, or formulation components, or formulation pH adjustments, that enhance the penetration of silibinin into these layers of the dermis and/or surfaces while maintaining the solubility of silibinin in the formulation and maintaining the stability of the entire formulation. This may include the specific inclusion of at least one “penetration enhancer.” The phrase “penetration enhancer,” as used herein, means any agent that enhances the penetration of silibinin into the skin, scalp, hair, or nails.

Topical formulations typically have a neutral, or slightly alkaline pH, whereas the topical compositions of this disclosure preferably have a pH below pH 7. At this pH, the silibinin is lipophilic and is more readily absorbed into the skin. The topical compositions of this invention may require pH adjustment of the final formulation in order to enhance the lipophilicity of the silibinin and enhance penetration of silibinin from the topical compositions of this disclosure into the skin. The pH of these compositions may be adjusted to a pH less than pH 7.0, or less than about pH 6.5, or less than about pH 6.0, or less than about pH 5.5. The pH of the composition may also be adjusted to a pH between about pH 1 and about pH 6.5. The pH of the composition may also be adjusted to a pH between about pH 2 and about pH 5.5. The pH of the composition may also be adjusted to a pH between about pH 4.0 and about pH 6.5. The pH of the composition may also be adjusted to a pH between about pH 4.5 and about pH 6.5. The pH of the composition may also be adjusted to a pH between about pH 5.0 and about pH 6.0. The pH of the composition may be adjusted to a pH of about pH 4.5, or a pH of about pH 5.0, or a pH of about pH 5.5, or a pH of about pH 6.0. Within this acidic pH range, the penetration of silibinin into the skin may be further enhanced. Adjustment of the final pH of the compositions of this disclosure may be achieved with a base or an acid, as needed.

In order to improve access or penetration of silibinin into the skin, hair, scalp, nails, or lips, which need protection or which are damaged and in need of treatment, vehicles which improve the penetration of silibinin through the outer layers of the skin are employed in the compositions of this disclosure.

Vehicle constituents which may improve the penetration or the preventative or therapeutic effects of the compositions of this disclosure include, but are not limited to: octyl methyl cinnamate, octyl salicylate, octinoxate, octisalate, avobenzone, octocrylene, polysorbates such as polysorbate 80 (TWEEN 80™), diethylene glycol ethers such as diethylene glycol monoethyl ether, azone (1-dodecylazacycloheptan-2-one), oleic acid, linoleic acid, propylene glycol, hypertonic concentrations of glycerol, lactic acid, glycolic acid, citric acid, and malic acid, urea, C₂-C₄ alcohols (such as ethanol and isopropanol), polyethylene glycol-3-lauramide, dimethyl lauramide, sorbitan trioleate, fatty acids, esters of fatty acids having from about 10 to about 20 carbon atoms, monoglycerides or mixtures of monoglycerides of fatty acids having a total monoesters content of at least 51% where the monoesters are those with from 10 to 20 carbon atoms, and mixtures of mono-, di- and tri-glycerides of fatty acids. Suitable fatty acids include, but are not limited to lauric acid, myristic acid, stearic acid, oleic acid, linoleic acid and palmitic acid. Monoglyceride permeation enhancers include glycerol monooleate, glycerol monolaurate, and glycerol monolinoleate.

Examples of penetration enhancers useful in the methods of this disclosure also include, but are not limited to those described in U.S. Pat. Nos. 3,472,931; 3,527,864; 3,896,238; 3,903,256; 3,952,099; 3,989,816; 4,046,886; 4,130,643; 4,130,667; 4,299,826; 4,335,115; 4,343,798; 4,379,454; 4,405,616; 4,746,515; 4,316,893; 4,405,616; 4,060,084; 4,379,454; 4,560,553; 4,863,952; 4,863,970; 4,879,275; 4,940,586; 4,960,771; 5,066,648; 5,164,406; 5,227,169; 5,229,130; 5,238,933; 5,308,625; 5,378,730; 5,420,106; 5,641,504; 5,716,638; 5,750,137; 5,785,991; 5,837,289; 5,834,468; 5,882,676; 5,912,009; 5,952,000; 6,004,578; the disclosures of which are incorporated herein by reference.

The compositions of this disclosure may include at least one sunscreening or sunfiltering agent sufficient to exhibit useful protection against UVA light, UVB light, or both, for prevention of damage to the skin from UV-light. UV radiation (UVA and UVB) play an important role in conditions such as premature skin aging, eye damage (including cataracts), and skin cancers. This radiation also suppresses the immune system, reducing the ability to fight off skin disease.

UVB ranges from 290 to 320 nm and is the chief cause of skin reddening and sunburn and damages the skin's more superficial epidermal layers. UVB plays a key role in the development of skin cancer and a contributory role in tanning and photoaging.

UVA is divided into two wave ranges, UVA I, which measures 340-400 nm and UVA II which extends from 320-400 nm. UVA is the dominant tanning ray. A tan results from injury to the skin's DNA, the skin darkens in an imperfect attempt to prevent further DNA damage and the DNA damage, or mutations, can lead to skin cancer. The skin damage from tanning has been shown to be cumulative over time.

As used herein, “sunscreen agent” includes both sunscreen agents and physical sunblocks. The sunscreen agents are liquid or crystalline substances at room temperature which are able to absorb ultraviolet radiation and emit the resulting energy in the form of longer wavelength radiation, for example as heat. The UV-A and UV-B sunscreen agents can be used individually, as well as in mixtures. The topical compositions of this disclosure may utilize a mixture of UV-A and UV-B agents. Suitable sunscreen agents may be organic or inorganic, hydrophilic or lipophilic.

Inorganic sunscreens useful in the compositions of this disclosure include metallic oxides such as titanium dioxide, zinc oxide, zirconium oxide, iron oxide, and mixtures thereof.

Organic sunscreen agents useful in this disclosure are selected from derivatives of dibenzoyl methane, cinnamic acid esters, diphenylacrylic acid esters, benzophenone, camphor, p-aminobenzoic acid esters, o-aminobenzoic acid esters, salicylic acid esters, benzimidazoles, symmetrically or unsymmetrically substituted 1,3,5-triazines, monomeric and oligomeric 4,4-diarylbutadienecarboxylic acid esters and -carboxylic acid amides, ketotricyclo(5.2.1.0)decane, benzalmalonic acid esters as well as any mixtures of the cited components. These organic sunscreen agents can be oil-soluble or water-soluble. Particularly preferred oil-soluble sunscreen agents are 1-(4-tert.-butylphenyl)-3-(4′-methoxyphenyl)propane-1,3-dione (Parsol™ 1789), 1-phenyl-3-(4′-isopropylphenyl)-propane-1,3-dione, 3-(4′-methylbenzylidene)-D,L-camphor, 4-(dimethylamino)-benzoic acid 2-ethylhexyl ester, 4-(dimethylamino)benzoic acid 2-octyl ester, 4-(dimethylamino)-benzoic acid amyl ester, 4-methoxycinnamic acid 2-ethylhexyl ester, 4-methoxycinnamic acid propyl ester, 4-methoxycinnamic acid isopentyl ester, 2-cyano-3,3-phenylcinnamic acid 2-ethylhexyl ester (Octocrylene), salicylic acid 2-ethylhexyl ester, salicylic acid 4-isopropylbenzyl ester, salicylic acid homomethyl ester (3,3,5-trimethyl-cyclohexyl salicylate), 2-hydroxy-4-methoxybenzophenone, 2-hydroxy-4-methoxy-4′-methylbenzophenone, 2,2′-dihydroxy-4-methoxybenzophenone, 4-methoxybenzmalonic acid di-2-ethylhexyl ester, 2,4,6-trianilino-(p-carbo-2′-ethyl-1′-hexyloxy)-1,3,5-triazine, dimethicodiethylbenzal malonate, dioctyl butamido triazone, 2,4-bis-[5-1(di-methylpropyl)benzoxazol-2-yl-(4-phenyl)-imino]-6-(2-ethyl hexyl)-imino-1,3,5-triazine and as well as any mixtures of the cited components.

Water-soluble sunscreen agents may include 2-phenylbenzimidazole-5-sulfonic acid, phenylene-1,4-bis-(2-benzimidazyl)-3,3′-5,5′-tetrasulfonic acid and their alkali metal, alkaline earth metal, ammonium, alkylammonium, alkanolammonium and glucammonium salts; sulfonic acid derivatives of benzophenone, preferably 2-hydroxy-4-methoxybenzophenone-5-sulfonic acid and its salts; sulfonic acid derivatives of 3-benzylidenecamphor, such as for example 4-(2-oxo-3-bornylidenemethyl)benzene sulfonic acid and 2-methyl-5-(2-oxo-3-bornylidene) sulfonic acid and their salts.

The compositions of the present invention will be administered topically, in the form of suitable formulations that are liquid, solid, or semi-solid. The topical compositions may be formulated in different topical products, including but not limited to, lotions, milks, mousses, serums, sprays, aerosols, foams, sticks, pencils, lipsticks, gels, emulsions, creams and ointments. The compositions may also be applied via a woven or nonwoven synthetic and/or natural fibered textile (i.e., wipe or towelette).

The compositions of the present disclosure are generally prepared by conventional methods known in the art of making compositions for topical application. Such methods typically involve mixing of the ingredients in one or more steps to a relatively uniform state, with or without heating, cooling, application of vacuum, and other methods. For example, silibinin may be dispersed in hydrophobic or anhydrous components of the topical formulations. All other formulation ingredients (except fragrance) may then be added to the resulting silibinin dispersion. The resulting mixture may be heated during or after the addition of the other formulation ingredients. The temperature of the mixture may be increased to approximately 70° C. to achieve a liquid suspension. The liquid suspension may be vortexed to disperse all formulation ingredients. Additionally or alternatively, the formulation ingredients may be mixed with an overhead mixer or homogenizer. As the formulation cools during mixing, one or more fragrances may be added when the preparation reaches approximately 50° C., while mixing. The preparation may become viscous as it approaches room temperature. The pH of the formulation may be adjusted to between pH 1 and pH 7 during any of the preparation steps or after completion of the ingredient mixing, by the addition of pH adjusting agents.

Cosmetic compositions formulated with composite particles of the present invention may contain a variety of optional components to enhance hypoallergenicity of the composition, and improve the physical properties, performance and the aesthetics of the compositions. Examples of good aesthetics may include luxurious creams and lotions that are light and nongreasy, have a smooth feel, spread easily, and/or absorb quickly. Other examples of good aesthetics include compositions that have a consumer-acceptable appearance (i.e. no unpleasant odor or discoloration present) and provide good skin feel. The aesthetic agent can be any one of fragrances, pigments, colorants, essential oils, skin sensates, and astringents. Examples of the suitable essential oils are olive oil, rose oil, palm oil, lavender oil, almond oil, Oenothera biennis (evening primrose) oil, clove oil, eucalyptus oil, peppermint oil and spearmint oil. Suitable aesthetic agents include clove oil, menthol, camphor, eucalyptus oil, eugenol, methyl lactate, bisabolol, witch hazel, and green tea extract.

The compositions of this disclosure may have additional desirable properties, including stability, long shelf-life, and the absence of significant skin irritation. To accomplish such additional benefits, the compositions of this disclosure may include agents such as antioxidants that promote composition stability, reduce skin irritation, and/or enhance the aesthetic appeal of the composition.

Topical compositions of this disclosure containing silibinin may also include conventional cosmetic adjuvants and additives including one or more preservatives (such as potassium sorbate), organic solvents, browning agents, antioxidants, stabilizers (such as sorbitol), lubricants (such as silicones, dimethicone), emollients, 21-alpha-hydroxy acids, demulcents, anti-foaming agents, moisturizing agents (such as cetyl alcohol), vitamins (such as niacinamide and/or tocopherol), fragrances (such as lavender oil), ionic or nonionic thickeners, surfactants (such as polysorbate 80), fillers, sequestrants, polymers, propellants, alkalinizing or acidifying agents, opacifiers, fatty compounds (e.g. oil, wax, alcohols, esters, fatty acids), colorants, or mixtures thereof or any other ingredient that may be used routinely in cosmetics or in sunscreen compositions. The optional components, when incorporated into the cosmetic compositions, should be suitable for use in contact with human skin without undue toxicity, incompatibility, instability, allergic response, and the like.

The International Cosmetic Ingredient Dictionary and Handbook (The Personal Care Products Council; 13th edition (Jan. 1, 2010)) describes a wide variety of nonlimiting cosmetic and pharmaceutical ingredients commonly used in the skin care industry, which are suitable for use in the compositions of the present invention. Examples of these classes include: abrasives, absorbents, aesthetic components such as fragrances, pigments, colorings/colorants, essential oils, skin sensates, astringents, etc. (e.g. clove oil, menthol, camphor, eucalyptus oil, eugenol, menthyl lactate, witch hazel distillate), anti-acne agents, anti-caking agents, antifoaming agents, antimicrobial agents, antioxidants, biological additives, buffering agents, bulking agents, chelating agents, chemical additives, colorants, cosmetic astringents, cosmetic biocides, denaturants, drug astringents, external analgesics, film forming polymers, lubricants, opacifying agents, pH adjusters, propellants, reducing agents, sequestrants, skin bleaching and lightening agents, skin conditioning agents, skin soothing and/or healing agents and derivatives, skin treating agents, thickeners, and vitamins and derivatives thereof.

The compositions of this disclosure may also contain one or more skin care agents in addition to silibinin. Other agents which can be incorporated into topical compositions of this disclosure include topical anesthetics (such as benzocaine, lidocaine, and benzyl alcohol), aloe vera, aloe barbadensis extract, aloe barbadensis leaf juice, retinoids, DNA repair enzymes, antibacterial agents (such as quaternary ammonium compounds, bacitracin, neomycin, polymyxin), zinc salts, and xanthines. More generally, known active ingredients compatible with the one or more topical formulation components of this disclosure, which have some utility in treating or attenuating various aspects of skin damage or discomfort, may be employed in topical compositions of this disclosure.

Emollients are substances which soften and soothe the skin. When applied to the skin in the compositions of this disclosure, emollients provide protection against dry skin. Emollients useful in the compositions of this disclosure may include more or more of hyaluronic acid, sodium hyaluronate, illipe butter, shea butter, shora seed butter, CERAPHYL 847™ (octyldodecyl stearoyl stearate), C₁₂₋₁₅ alkyl benzoate, pentaerythrityl tetraisostearate, and/or diisopropyl adipate.

Suitable surfactant/emulsifying agents include glyceryl stearate, ceteareths, ceteths, laneths, laureths, isoseareths, steareths, cetyl alcohol, deceths, dodoxynols, glyceryl palmitate, laneths, myreths, nonoxynols, octoxynols, oleths, PEG-castor oil, poloxamers (e.g., poloxamer 407), poloxamines, polysorbates (including Polysorbate 80), sodium laurate, ammonium laureth sulfate, sodium laureth sulfate, sodium lauroyl sarcosinate, sodium lauroyl taurate, sodium lauryl sulfate, sodium methyl cocoyl taurate, sodium methyl oleoyl taurate, sodium nonoxynol sulfate, sodium cetyl sulfate, sodium cetearyl sulfate, sodium cocoate, sodium cocoyl isethionate and sodium cocoyl sarcosinate. Additional suitable surfactant/emulsifying agents are listed in the International Cosmetic Ingredient Dictionary and Handbook, The Personal Care Products Council; 13th edition (Jan. 1, 2010). These compounds may also function to enhance penetration of silibinin into the skin.

Examples of preservatives suitable for use in the compositions of this disclosure include sorbates and salts thereof (such as potassium sorbate), imidazolidinyl urea, diazolidinyl urea, phenoxyethanol, methylparaben, ethylparaben, propylparaben, phenoxyethanol, capryl glycol, ethylhexylglycerin, hexylene glycol, and combinations of these preservatives.

Examples of thickening agents suitable for use in the compositions of this disclosure include isopropyl myristate, isopropyl palmitate, isodecyl neopentanoate, squalene, mineral oil, C₁₂-C₁₅ benzoate, and hydrogenated polyisobutene.

Additional embodiments of the compositions of this disclosure may include permutations of the various ingredients described above. Therefore, topical formulations including such permutations of the ingredients disclosed herein are expressly within the scope of this disclosure.

This disclosure also includes medicaments for the treatment or prevention of skin damage, as well as methods for preparation of such medicaments. Such methods involve a combination of, and effective amount (or combined effective amount) of, silibinin with one or more of the agents described above with a carrier useful for topical administration.

The compositions of this disclosure are robust to the presence or the various permutations of the ingredients and medicaments disclosed herein, while maintaining the biological activities of silibinin and enhanced skin penetration of silibinin described above.

Methods of Administering Compositions of this Disclosure

The methods of using these compositions includes topically applying a therapeutically effective amount of a composition of this disclosure to a topical area of a subject for the treatment, prevention and/or the reduction of risk of multiple dermatologic conditions as well as skin aging, including but not limited to, facial redness/erythema associated with acne rosacea and skin aging; xerosis/dry skin; dyspigmentation; traumatic bruising/post-procedure bruising/senile purpura; skin wounds; chemically-induced skin damages caused by cytotoxic and vesicant chemical warfare agents such as mustard gas, or sulfur mustards and nitrogen mustards; chemically-induced skin damages such as contact dermatitis, irritant dermatitis, and dermatitis/atopy, skin inflammation such as psoriasis, diaper dermatitis, atopic dermatitis; skin photo aging such as wrinkles and dyspigmentation caused by ultraviolet radiation, photo carcinogenesis such as actinic keratosis/non-melanoma skin cancer, environmentally-induced skin damages such as UVA and UVB-induced skin damages such as sunburn/tanning/skin thickening/wrinkles; skin oxidative damage; environmentally-induced skin damages such as radio dermatitis/fibrosis associated with radiotherapy. These methods include contacting the skin, scalp, hair, lips or nails, or other target sites of the subject with a composition of this disclosure described above.

In the present disclosure, the term “subject” means a mammal, preferably a human. The subject can be a human consumer or patient.

The term “therapeutically effective amount” means that amount of the composition that provides a therapeutic benefit in the treatment, prevention, or management of one or more skin conditions.

The compositions of this disclosure are also useful for the regulation of skin conditions, including the signs of skin aging. The regulation of a skin condition involves improving skin appearance and/or feel, and may include delaying, minimizing, preventing, and/or ameliorating the signs of skin aging. This may include treating or minimizing all outward visible and tactile manifestations due to skin aging, including but not limited to wrinkles, skin lines, skin spots, skin discoloration, skin roughness, and loss of skin firmness and/or elasticity. Such signs may be caused or induced by intrinsic or extrinsic factors, e.g., chronological aging and/or environmental damage including, but not limited to sunlight, UV radiation, smoke, ozone, and pollutants.

Silibinin has anti-oxidant, anti-inflammatory, and anti-aging effects on the skin. Therefore, the topical silibinin compositions of this disclosure may be used to prevent or treat oxidative damage to the skin, as well as photoaging caused by ultraviolet radiation. Silibinin has anti-cancer and anti-inflammatory effects on the skin. Therefore, the topical silibinin compositions of this disclosure may be used to prevent or treat skin inflammation, photocarcinogenesis (such as actinic keratosis/non-melanoma skin cancers), chemically-induced skin damages (such as contact dermatitis), irritant dermatitis, dermatitis/atopy, as well as facial redness/erythema associated with acne rosacea and skin aging. Silibinin promotes DNA repair and wound healing in the skin. Therefore, the topical silibinin compositions of this disclosure may be used to prevent or treat skin wounds, environmentally-induced skin damage (such as UVA and UVB-induced skin damage including sunburn/tanning/skin thickening), chemically-induced skin damage caused by cytotoxic and vesicant chemical warfare agents (such as, sulfur mustard, and nitrogen mustard), environmentally-induced skin damage (such as radiodermatitis/fibrosis associated with radiotherapy), as well as traumatic bruising/post-procedure bruising/senile purpura. Silibinin also moisturizes and whitens and brightens the skin. Therefore, the topical silibinin compositions of this disclosure may be used to prevent or treat xerosis, dry skin, as well as dyspigmentation of the skin.

The compositions of this disclosure are useful for topical application i.e., on the exterior surface of the body, and specifically on skin, hair, scalp, lips and nails. This disclosure provides methods of treating or preventing damage to the skin that may result from age, or exposure to conditions or agents that damage the skin, by administering to a subject susceptible to such damage or having sustained such damage, a therapeutically effective amount of a composition of this disclosure. The compositions may be administered after exposure of the individual to skin damaging agents or conditions, such as UV-light. The compositions are preferably applied in a thin layer on the skin covering the area to be treated or protected. The compositions may be rubbed until the composition is no longer visible on the surface of the skin or other target area. This may enhance the penetration of the silibinin into the epidermis or dermis.

This disclosure also provides methods of treating, preventing, or managing dermatological conditions by administering to a patient a therapeutically effective amount of a composition of this disclosure while substantially avoiding irritation to the skin, scalp, hair, or nails. The compositions may also be administered in concentrations that are safe and effective for products that remain in contact with the skin for long periods of time. The compositions may also be administered in high concentrations for administration followed by removal shortly thereafter.

Therapeutically effective amounts of the compositions of this disclosure will, of course, depend on the severity of the skin damage being treated or the anticipated damage that the skin is being protected against, as well as the weight and general state of the subject. Typically, dosages used in vitro may provide guidance in the amounts useful for in situ administration of the pharmaceutical composition, and animal models may be used to determine effective dosages for treatment of particular disorders.

The methods of treating skin damage to a subject by applying a composition of this disclosure, may include administering from about 15 mg to about 6,000 mg silibinin daily, per square inch of skin, to a subject in need of such treatment.

The methods of preventing skin damage to a subject by applying a composition of this disclosure, may include administering from about 0.001 mg to about 1.8 grams silibinin daily, per square inch of skin, to a subject in need of such preventative treatment. In one embodiment of the method of preventing skin damage by applying a composition of this disclosure, from about 0.010 mg to about 30 mg silibinin is administered daily, per square inch of skin, to the subject using a composition of this disclosure. In one embodiment of the method of preventing skin damage by applying a composition of this disclosure, at least about 0.03 mg silibinin is administered daily, per square inch of skin, to a subject using a composition of this disclosure. In one embodiment of the method of preventing skin damage by applying a composition of this disclosure, about 0.1 mg silibinin is administered daily, per square inch of skin, to a subject using a composition of this disclosure. In one embodiment of the method of preventing skin damage by applying a composition of this disclosure, about 1 mg silibinin is administered daily, per square inch of skin, to a subject using a composition of this disclosure.

In methods and uses of the compositions of this disclosure for prevention of skin damage, or protection of skin from damage, the compositions may be applied to the subject in need of such protection prior to exposure to conditions or agents that damage the skin, such as UV-light, or other sources of radiation. The composition may be applied 12 hours or less, or 5 hours or less, or more preferably 2 hours or less, prior to exposure of the subject to the skin damaging agent or condition.

In methods and uses of this disclosure for treating skin damage, a composition of this disclosure may be applied to the subject in need of such treatment after exposure of the subject to a skin damaging agent or condition. The composition may be administered as soon as possible after exposure to the skin damaging agent. Treatment is often administered much later, when detrimental symptoms of skin damage are noted.

Topical application of the compositions of this disclosure may be more than about once, twice, three times, four times, five times, six times or seven times per week, or if necessary, even more than once, twice, three times, four times, five times, six times or seven times per day.

Methods of this disclosure for the treatment of the skin of a subject by topical administration of an effective amount of a composition of this disclosure, may include administration of the composition an average of once per day; or an average of twice per day; or an average of three times per day; or more than about three times per day. The composition may be administered an average of about twice per day, in the morning upon rising and in the evening before retiring. The composition may be applied and left on the skin. Alternatively, the composition may be applied and removed, for example, by washing the skin. The composition may also be applied by spot application, for example, directly to areas where necessary, or may be applied generally, to cover an entire skin region.

The compositions of the present invention will be administered topically, in the form of suitable formulations both liquid (such as gel, lotions, milks, emulsions, serums, foams and the like) and solid or semi-solid (such as creams, ointments, lipsticks, and the like). The compositions of the present invention may also be integrated directly into packaged bandages for convenient application. The bandages of this disclosure come with the compositions of this disclosure pre-applied to allow for easy administration to a subject. These formulations will be prepared with suitable excipients, such as emollients, moisturizers, thickening agents, emulsifiers, dyes, fragrances, and the like, as described above and known to those skilled in the pharmaceutical arts.

### Kits

In still another aspect, the present disclosure provides kits for the treatment or prevention of skin or to produce a desired cosmetic result. These kits comprise the compositions described herein, in a container or containers which are held in suitable packaging. These kits may further contain instructions teaching the use of the kit components according to the various methods described below. Such kits may also include information, such as scientific literature references, package insert materials, cosmetic trial results, and/or summaries of these and the like, which indicate or establish the activities and/or advantages of the composition and/or recommended methods of applying the topical formulations of this disclosure. Such information may be based on the results of various studies, for example, studies using experimental animals involving in vivo models and studies based on human cosmetic or clinical trials. Kits described herein can be provided, marketed and/or promoted to health care providers (e.g., dermatologists and other physicians), skin care appearance care providers, including cosmetologists, hair stylists, and the like. Kits for cosmetic use may also be provided, marketed and/or promoted directly to consumers.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims. All references cited herein are incorporated in their entirety by reference.

This disclosure now being described may be more readily understood by reference to the following examples, which are included merely for the purposes of illustration of certain aspects of the embodiments of the present invention. The examples are not intended to limit this disclosure, as one of skill in the art would recognize from the above teachings and the following examples that other techniques and methods can satisfy the claims and can be employed without departing from the scope of the claimed invention.

### EXAMPLES

### Example 1: Demonstrating the Preparation of One Silibinin-Containing Formulation of this Disclosure

A topical formulation is prepared containing:

| INGREDIENT | percent (w/w) |
|---|---|
| Cetyl alcohol | 6.0 |
| Sorbitol | 5.0 |
| Glyceryl stearate | 3.5 |
| Niacinamide | 2.0 |
| Silibinin | 2.0 |
| Polysorbate 80 | 1.5 |
| Tocopherol | 1.0 |
| Dimethicone | 1.0 |
| Aloe barbadensis leaf juice | 1.0 |
| Fragrance (lavender/chamomile) | 1.0 |
| Preservative composition containing | 0.75 |
| phenoxyethanol, caprylyl glicol, | |
| ethylhexylglycerin, and hexylene glycol | |
| Potassium sorbate | 0.10 |
| Sodium hyaluronate | 0.05 |
| Water | qS 100% |

This composition forms a topical oil-in-water emulsion and the pH is adjusted to final pH between pH 5 and pH 6, after mixing. This formulation shows high penetration of silibinin into skin and offers superior protection against radiation-induced skin damage.

### Example 2: Demonstrating the Efficacy of the Topical Silibinin Compositions in Animal (Mouse) Models

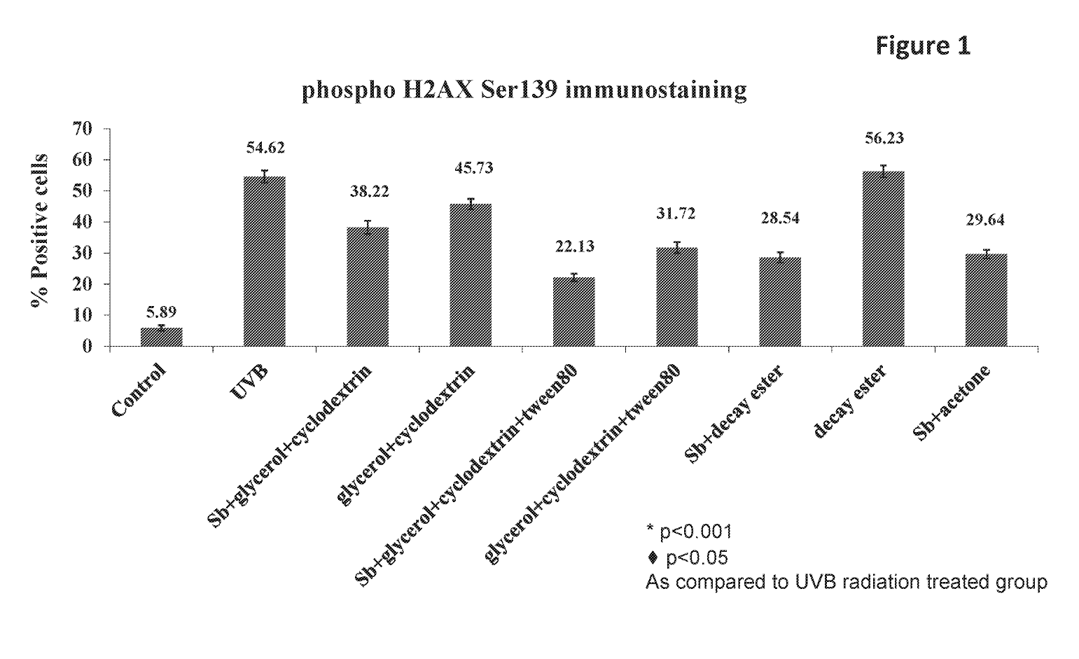

DNA damage, as measured by phospho H2AXser139, is commonly used as a measure of DNA damage. Topical application of a silibinin/sunblock formulation in mice reduced DNA damage caused by UVB irradiation. Following different topical treatments and a single UVB exposure at 180 mJ/cm², mice were sacrificed and skin tissue samples were collected, followed by immunohistochemistry staining for phospho-H2A.X and TUNEL staining for apoptotic cells. FIG. 1 shows that several different silibinin-containing compounds significantly (>2 X) decreased DNA damage in irradiated mouse skin cells (Sb=silibinin). While control animals showed about 55% positive cells following UVB radiation, protection afforded by a silibinin formulation of this disclosure resulted in a significant decrease in positive cells, to about 22% positive cells. These data demonstrate that topical application of silibinin formulations of this disclosure on mice reduces DNA damage caused by UVB irradiation.

A. Silibinin Penetration into Mouse Skin from Topical Compositions of this Disclosure.

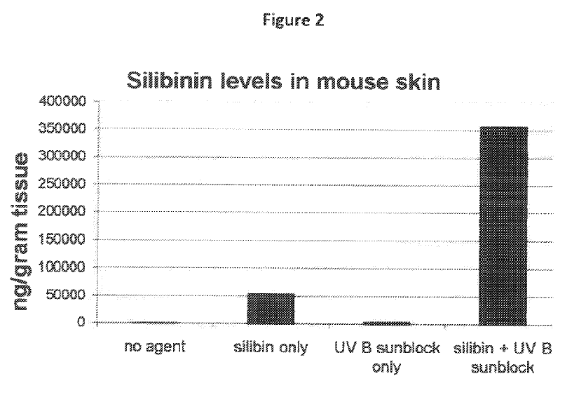

The topical silibinin compositions of this disclosure have superior penetration in mouse skin. This was demonstrated by topically applying either silibinin alone (not lipophilic), UVB blockers alone, or silibinin+UV blockers to mice (4 mice/group). Eight hours later, dermis and epidermis was isolated and silibinin levels were analyzed by HPLC. As FIG. 2 shows, the formulation with silibinin+UV blockers in the formulation of the present invention showed surprisingly high levels of penetration in the skin: on average, 7-fold higher than topical silibinin administered alone.

B. Topical Application of Silibinin Compositions of this Disclosure on Mice Reduces Thymine Dimer Formation Caused by UVB Irradiation.

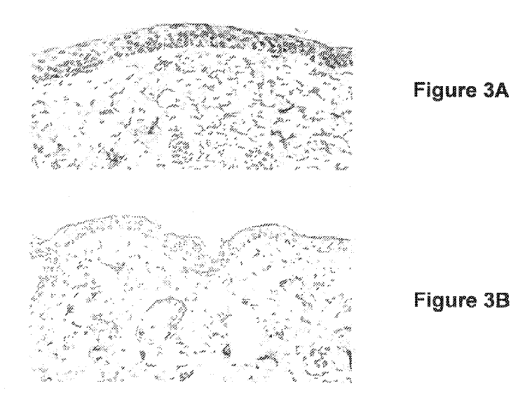

This was demonstrated using skin samples from the same mice used above for the skin penetration studies. The harvested skin was analyzed for thymine dimer formation, a rapid photoreaction associated with skin photodamage. FIGS. 3A and 3B show two magnified images of UVB-irradiated mouse skin (400× magnification). FIG. 3A is mouse skin irradiated with UVB radiation with no silibinin, and no UV protectant. FIG. 3B is mouse skin irradiated with UVB radiation after application of a composition of this disclosure containing silibinin and a UV protectant. As shown in FIGS. 3A and 3B, immunohistochemical staining of the skin using red chromogen, 3-Amino-9-ethylcarbazole (AEC) reveals a dramatic reduction in the presence of thymine dimers, presumable due to enhanced thymine-dimer excision rates after skin exposed to topical silibinin compositions of this disclosure.

C. Experimental Optimization of Silibinin Penetration into Epidermis and Dermis in Animal Studies, in the Presence or Absence of UVA/UVB Sunscreens.

Mice are divided into 5 groups as follows:
1—control (DMSO only, negative control),
2—(silibinin in DMSO, positive control for silibinin penetration though unsuitable for human application),
3—formulation base without silibinin,
4—formulation base+silibinin and
5—formulation base+silibinin+UV sunscreens);

Ten mice/group (5 mice/group/time point) will be treated topically as indicated. Subsequently, at either 8 hr or 16 hr following treatment, shaved skin tissues are collected, wiped several times to remove any remainder formulation from the skin, and then separated into epidermis and dermis. Briefly, shaved skin is dried for 5 minutes, placed on 0.25% trypsin, dermis side down, and floated overnight at 4° C. The next day skin is lifted off the trypsin and the dermis layer is peeled away, leaving the epidermis behind; both layers are snap frozen. Silibinin levels in the skin layers are then assessed.

The optimal topical compositions have enhanced penetration into both dermal and epidermal skin layers as the epidermis is where actinic keratosis (AK) and squamous cell carcinomas (SCC) initiate, and the dermis is the site of oxidative damage to collagen and other proteins.

D. Phase II Human Clinical Trials Using Optimized Silibinin/Sunscreen Compositions, Includes Testing for Allergy, Sun Protections Factor (Spf), Ultraviolet A and B (UVA and UVB) Protection, and Clinically Relevant Biomarkers that Give Rise to AKs and/or SCCs.

Normal adult human subjects (n=30) are exposed over a 3-week interval using Finn chambers patch tests of:

a) base topical formulation vehicle,

b) 4% silibinin base vehicle

c) 4% silibinin in base vehicle with broadband UV sunscreen(s) and

d) a positive irritant control (sodium lauryl sulfate).

The application sites are scored using a grading scale ranging from 0 (no reaction) to 4 (severe erythematous reaction) for possible cutaneous erythematous reactions 30 minutes after removal of the patches during the induction phase, and at 48, 72, and 96 hours (contact sensitization potential) after the challenge application and on days 2 to 22 (cumulative irritation potential). Normal adult subjects (n=30) will be exposed to graded broadband UVB on non-sun exposed skin (right buttock) to determine MEDs. MEDs are used to determine SPF per standard protocols. FDA guidelines of a 4-star rating system for UVA protection are followed; the in vivo Persistent Pigment Darkening (PPD) method will be used for measuring UVA protection, similar to the SPF method of measuring UVB light protection. Normal adult subjects will be treated topically as indicated prior to UV treatment. Subsequently, at either 8 hr or 16 hr (n=5), shaved skin tissues are collected, wiped several times to remove any remainder formulation from the skin, separated into epidermis and dermis, and processed for immunohistochemical staining of relevant biomarkers: cyclobutane pyrimidine dimers (CPDs, including thymine dimers) and silibinin levels.

### Example 3: Stability Testing

Stability testing was conducted on the silibinin formulation of this disclosure described in Example 1. Aliquots of the 2% silibinin formulation (drug loading of approximately 2.0±0.1%) were stored at room temperature and at 40° C. for two months. Each aliquot was sampled four times over the two-month period and the relative concentration of silibinin was determined via HPLC/UV analysis.

The sampling and testing protocol included introducing four samples of approximately 200 mg each the formulations into individual glass vials. These samples were diluted with 5.0 mL methanol, and vortex mixed into solution. These solutions were mixed and 100 μL aliquots were added to 4900 μL of MeOH (Dilution 1). Thereafter, 500 μL of Dilution 1 was mixed with 4500 μL of MeOH (Dilution 2). These dilutions were analyzed via LC/MS-MS analysis. For the HPLC/UV analysis, the initial methanolic solution sample (100 μL) was diluted with 1900 μL MeOH and 50 microL injections analyzed by HPLC/UV (290 nm). HPLC was performed on long 18C column, 5% solvent A (water, ammonium acetate (10 mM), and formic acid (0.1%) for min, ramped to 95% solvent B (MeOH:Acetonitrile) at 19 min, held at 95% for 13.5 min, and then returned to 5% at 36 min, and held at 5% for 4 min; 40 min total run time.

Additional stability testing was conducted on the formulation packaged in 3-ounce white tubes with dispensing pump, stored in controlled temperature chambers at 5° C., 25° C., and 40° C. Physical testing of the aliquots included evaluation of color, odor, appearance, pH, and viscosity. Microbiological testing of the aliquots included testing for total aerobic count and total yeast and molds. Preservative efficacy testing included an evaluation of antimicrobial effectiveness testing.

### A. Silibinin Concentration

The samples maintained at 40° C. displayed no statistical difference in silibinin content compared to the room temperature samples in any aliquot, or at any time point. These data demonstrate that the silibinin formulations of this disclosure maintain the full silibinin content for at least two months at room temperature and at elevated temperature with no statistical settling, precipitation, or other loss of silibinin content.

### B. Physical Stability

Following three cycles of Freezing/Thawing, the tested formulations showed ingredient separation. The formulation also showed darkening/browning on surface and around its walls after three months at 40° C. In all aliquots tested, the pH remained within specified limits. viscosity at 5° C. increased from the initial reading of 23,950 centipoise (cps) to 45,250 cps. Viscosity at 25° C. decreased from the initial reading of 23,950 cps to 17,000 cps. Viscosity at 40° C. increased from the initial reading of 23,950 cps to 33,250 cps.

No ingredient separation was observed in any of the samples maintained at 5° C., 25° C., or 40° C.

### C. Microbiological Stability

The formulations all passed microbiological testing at 3 months at 40° C.

### D. Preservative Efficacy Testing

The preservative was shown to be effective in exerting antimicrobial effectiveness and in maintaining the sterility of the product.

These data from the samples maintained 3-months at 5° C., 25° C., and 40° C. indicated that the formulations of this disclosure are physically, chemically, and microbiologically stable. Based on this standard, accelerated stability testing, a formulation stability of 2 years is expected, an expiration date of 2 years can be assigned. A recommendation to avoid repeated freeze thawing should be included on the formulation packaging.

### Example 4: Skin Penetration

Five formulations, each containing 1% silibinin, were formulated with components and amounts according to the following table (values are mg content in 20 g total formulation weight):

| | 1 | 2 | 3 | 4 | 5 |
|---|---|---|---|---|---|
| Vitamin E | 20 | 20 | 20 | 20 | 20 |
| Cetyl alcohol | 736 | 736 | 736 | 736 | 736 |
| Glyceryl monostearate | 304 | 304 | 304 | 304 | 304 |
| Dimethicone | 384 | 384 | 384 | 384 | 384 |
| Tween 80 | 200 | 200 | 200 | 200 | 200 |
| Octinoxate | 1500 | 750 | 375 | 150 | 0 |
| Octisalate | 1000 | 500 | 250 | 100 | 0 |
| silibinin | 200 | 200 | 200 | 200 | 200 |
| SDS | 160 | 160 | 160 | 160 | 160 |
| Methyl paraben | 20 | 20 | 20 | 20 | 20 |
| Propyl paraben | 10 | 10 | 10 | 10 | 10 |
| water | qs | qs | qs | qs | qs |
| 1M HCl | 120 | 120 | 120 | 120 | 120 |
| Sorbitol | 1000 | 1000 | 1000 | 1000 | 1000 |

In these formulations, the level of sunscreen varies:

Formulation #1 had octinoxate 7.5%, and octisalate 5%;

Formulation #2 had octinoxate 3.75%, and octisalate 2.5%;

Formulation #3 had octinoxate 1.875%, and octisalate 1.25%;

Formulation #4 had octinoxate 0.75%, and octisalate 0.5%;

Formulation #5 had octinoxate 0%, and octisalate 0%.

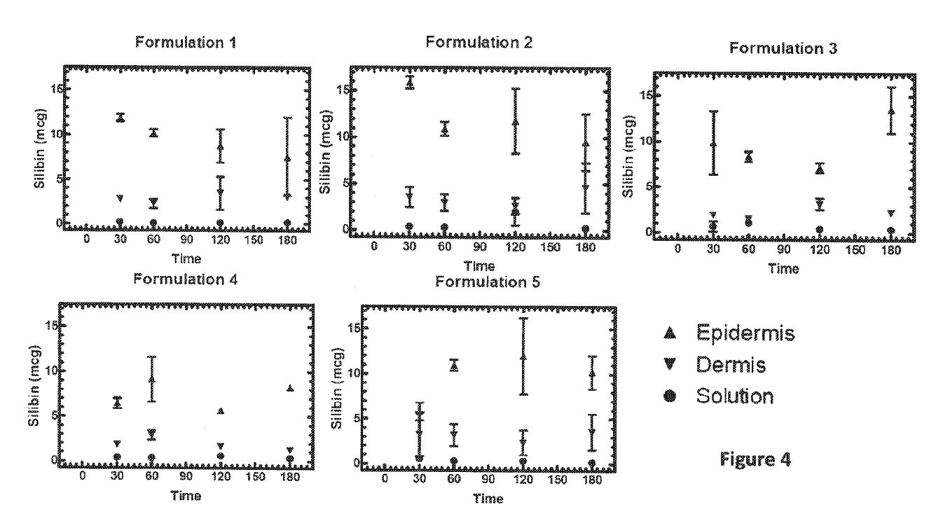

Pig ears mounted on a Franz cell (with buffer flowing underneath to simulate blood flow), were treated with each of the five formulations. The dermis and the epidermis were separated and evaluated for silibinin content at various timepoints. Two samples were run per timepoint. At each timepoint, silibinin levels in dermis, epidermis, and solution (buffer used to simulate blood flow) were quantified by Mass Spectrometry. Silibinin concentrations in epidermis, dermis, and solution at time points between 0 and 180 minutes for each of the five formulations is shown in FIG. 4.

The data showed consistent accumulations of silibinin after 30 minutes in both epidermis and dermis. At later time points, about 3-fold more silibinin was detected in the epidermis, compared to the dermis, and a trend toward increasing silibinin in the dermis was observed. Surprisingly, virtually no silibinin was detected in the solution, indicating that very little silibinin is absorbed and transferred to “blood” in this in vitro animal skin model, in the first 3 hours, indicating that silibinin stays in the skin where we want it. An additional surprising finding is that the level of sunscreen in these tested formulations had no effect on silibinin penetration levels in the skin layers in this in vitro animal skin model, indicating that it is the formulation which allows penetration into the skin. Finally, silibinin concentrations were detected in the skin between 30 minutes and 3 hours in these tests with an observed trend toward increasing silibinin in the dermis (and a consequent decrease in the epidermis) at later time points.

### Example 5: Silibinin Efficacy Testing—Sulfur Mustard Model

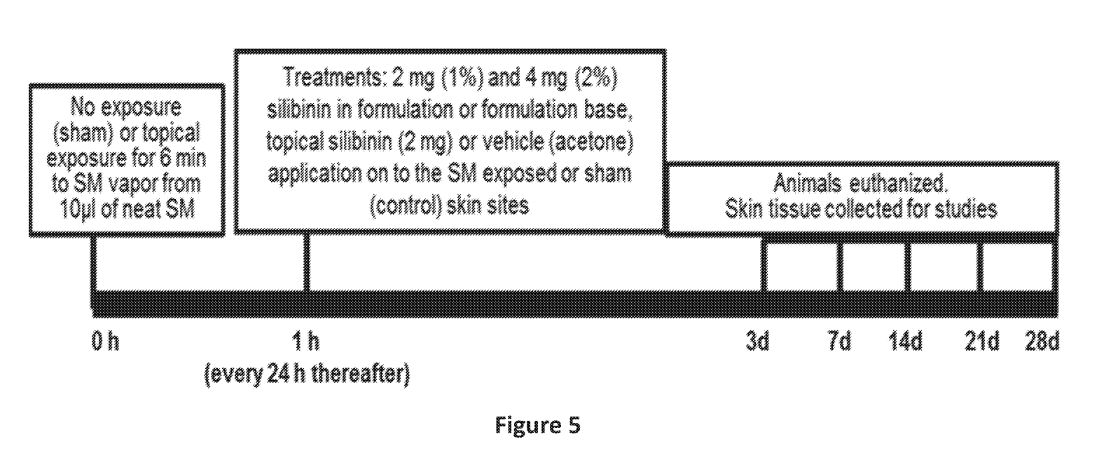

The formulations of this disclosure described in Example 1 were tested for efficacy in treating damage to skin in mice exposed to sulfur mustard (SM) or control (C). SM exposure on the dorsal skin of SKH-1 hairless mouse is a model of skin damage that causes strong skin toxicity, including edema, inflammation, and vesication. Mouse skin was exposed to neat SM vapor using a vapor cup for 6 minutes (exposure and treatment protocol outlined in FIG. 5). Treatments commenced at 1h after SM exposure, and every 24 hours thereafter for 28 days. Treatments included:

1. Control (sham, no sulfur mustard exposure)

2. formulation base alone (FB)

3. 1% silibinin in acetone (SB/Ace)

4. 1% silibinin formulation of Example 1 (SB-1% F)

4. 2% silibinin formulation of Example 1 (SB-2% F)

At 1, 3, 7, 21 and 28 days post-exposure, mice were euthanized and dorsal skin tissue punches from the control and exposed sites of all the study groups were collected and either: 1) snap frozen using liquid nitrogen, 2) fixed in 10% phosphate-buffered formalin, or 3) fixed in Bouin's solution. Fixed skin tissue samples were processed and skin sections were stained, and the frozen tissue was subjected to lysate preparation and protein isolation for western blot analysis. The tissue was evaluated for injury endpoints including vesication, myeloperoxidase and COX-2 levels, and collagen changes.

### A. Microvesication

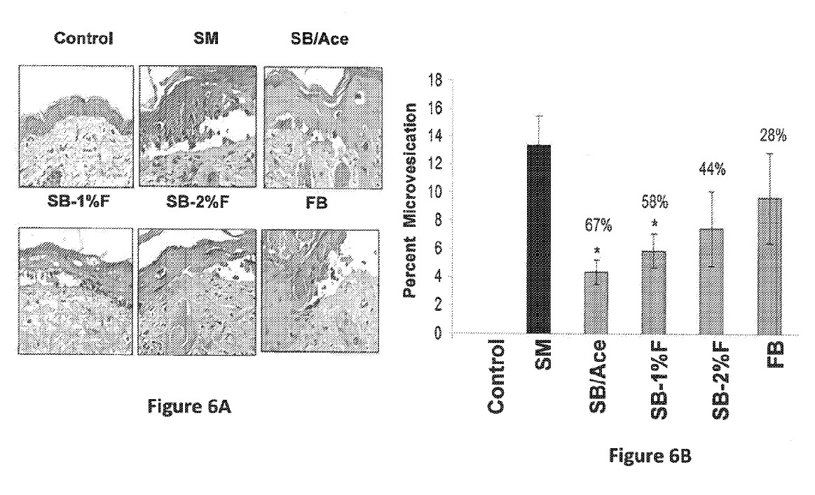

SM exposure caused inflammation-related histopathological alterations and microvesication in the mouse skin. FIG. 6A shows representative stained skin sections from control, sulfur mustard (SM), silibinin in acetone (SB/Ace), 1% silibinin formulation (SB-1%), and 2% silibinin formulation (SB-2%); arrows indicate separation in dermis/epidermis indicative of microvesication under ×400 magnification. FIG. 6B shows the percent microvesication recorded in a skin section. Percentages shown above each bar indicate percentage decrease in SM-induced microvesication following treatments These data show that 1% silibinin in acetone provided the best treatment of the skin exposed to SM; both 1% and 2% silibinin formulations of this disclosure were effective in the treatment of SM-induced microvesication in the mouse skin tissue. The formulation base alone provided weak and non-significant (less than 30%) effect in the treatment of microvesication in the SM-exposed mouse skin.

### B. Myeloperoxidase Levels

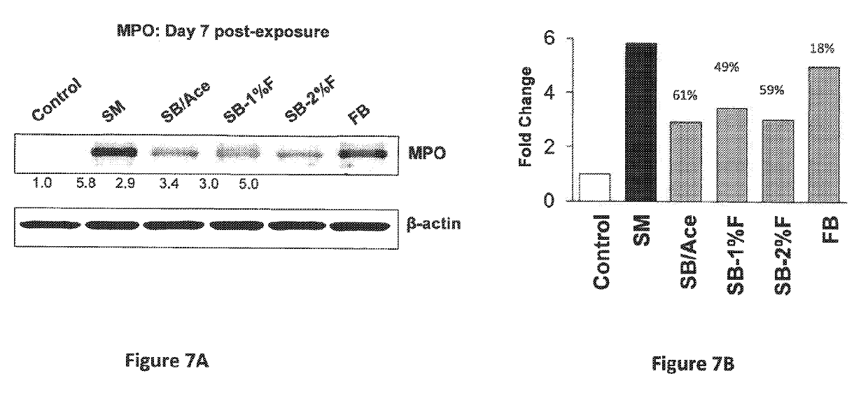

Tissue myeloperoxidase (MPO) activity/levels, an indicator of neutrophil infiltration, is an established biomarker of SM-induced skin toxicity in the mouse models. We evaluated the MPO levels in the skin tissue using western blot analysis. The MPO levels drastically increased following SM exposure (FIG. 7A). MPO levels were reduced in the skin treated with silibinin in acetone and the 1% and 2% silibinin formulations. A minor reduction in MPO levels was seen in the formulation control. These results are shown graphically in FIG. 7B, based on the fold changes observed in the representative western blot; percentages above bars indicate percent decrease in MPO levels following sulfur mustard exposure and the indicated sillibinin (or formulation control) treatment.

### C. COX-2 Levels

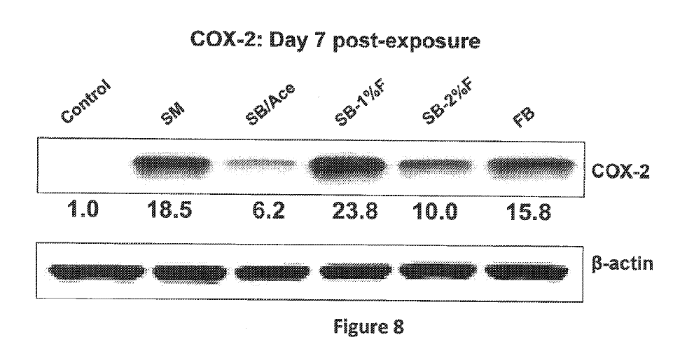

Similarly, COX-2, another marker of inflammation, was also determined in these skin samples. Skin sections from the hairless mice showed strong increase in the COX-2 levels following SM exposure FIG. 8. A strong reduction in SM-induced increase in the COX-2 levels was seen following treatment with 1% silibinin in acetone, and in the 2% silibinin formulation.

### D. Collagen Changes

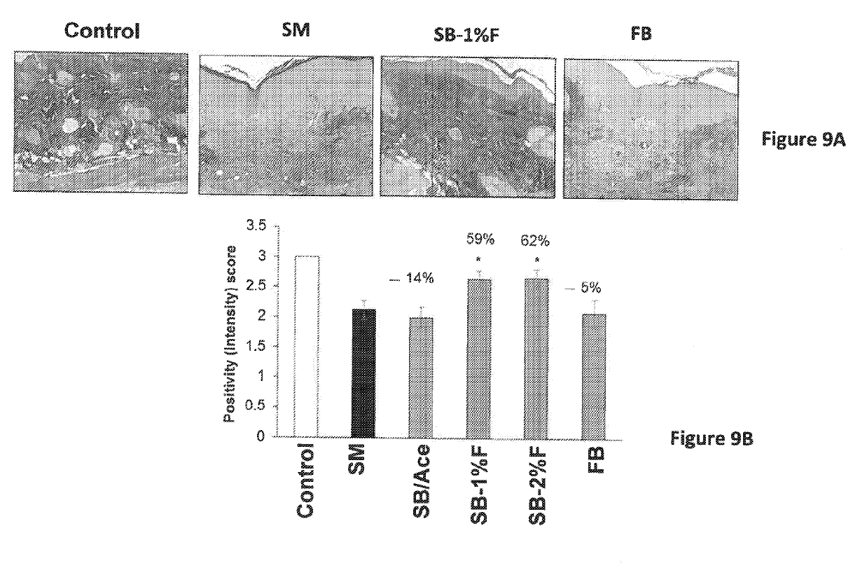

Collagen changes following SM exposure was also determined in these skin samples using trichrome staining of collagen fibers, which was quantified using positivity (intensity) score. Collagen degradation and fragmentation (Lighter trichrome staining) was observed following SM exposure Silibinin formulation treatments (of this disclosure) reversed these effects as seen by darker collagen staining (FIG. 9A). Percentages above bars indicates percent collagen recovery compared to SM exposure.

These data demonstrate that treatment with the silibinin formulations of this disclosure, at concentrations of 1% and 2%, were effective in reversing (>40%) of sulfur mustard (SM)-induced microvesication, myeloperoxidase and COX-2 levels, and collagen degradation.

### Example 6

Chemical warfare agent sulfur mustard inflicts delayed blistering and incapacitating skin injuries. To identify effective countermeasures against HD-induced skin injuries, efficacy studies were carried out employing sulfur mustard analog 2-chloroethyl ethyl sulfide (CEES)-induced injury biomarkers in skin cells. The data demonstrate strong therapeutic efficacy of silibinin in attenuating CEES-induced skin injury and oxidative stress. In skin cells, silibinin treatment 30 min before, 30 after, 60 min before, 60 after CEES exposure caused a significant prevention or reversal in CEES-induced decrease in cell viability, apoptotic and necrotic cell death, DNA damage, and an increase in oxidative stress.

### A. MTT Assay for Viability of Skin Epidermal Cells and Fibroblasts

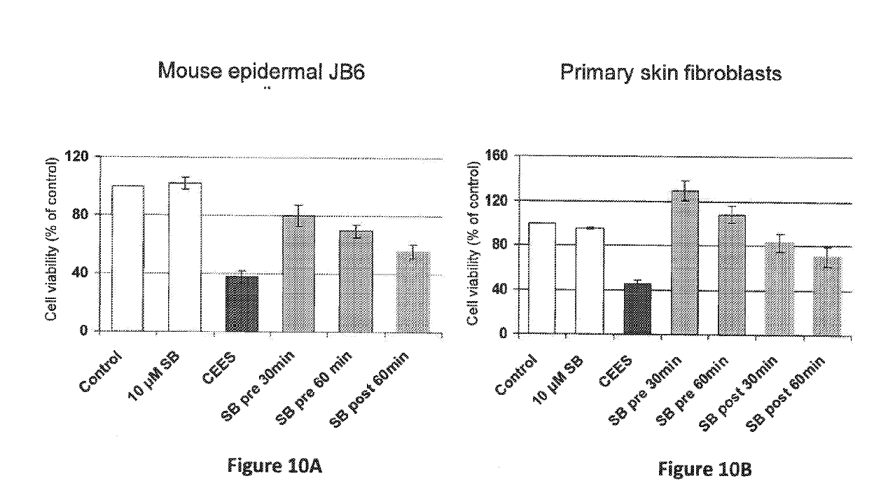

MTT assay (with 1 mg/mL of MTT; Sigma-Aldrich) for cell viability was conducted at 24 or 48 h following the desired exposures and treatment of cells. Briefly, at the desired time point the culture medium was removed, MTT was added to the cells in serum free medium for 4 h at 37° C. The MTT solution was then removed and absorbance was read at 540 nm following the addition of 100 mL DMSO. Hoechst-propidium iodide (PI) staining, was carried out after 24 h of above mentioned exposures and treatment by staining cells with 10 mL of PI (1 mg/mL) and Hoechst 33342 (1 mg/mL; Sigma) at a ratio of 3:1. At the end of each treatment time, both floaters and attached cells were collected, washed twice with 16PBS, kept on ice and stained with Hoechst-PI dye at a ratio of 3:1. For Hoechst 33342, the excitation wavelength is 350 nm and emission at 461 nm and for PI excitation is at 535 nm and emission at 617 nm. Qantification of cells was performed in triplicate for each treatment and 200 cells per sample were counted in different fields (at least 10 random fields per sample) to score for percent live, apoptotic and necrotic cells, using a fluorescent microscope. Silibinin treatment at 10 μM concentration 30 min and 60 minutes before and after CEES exposure for 24 h results prevention or reversal of CEES-induced decrease in cell viability in mouse epidermal JB6 cells, and primary skin fibroblasts (FIGS. 10A and 10B, respectively).

### B. BrdU Assay for DNA Synthesis in the Skin Epidermal Cells and Fibroblasts

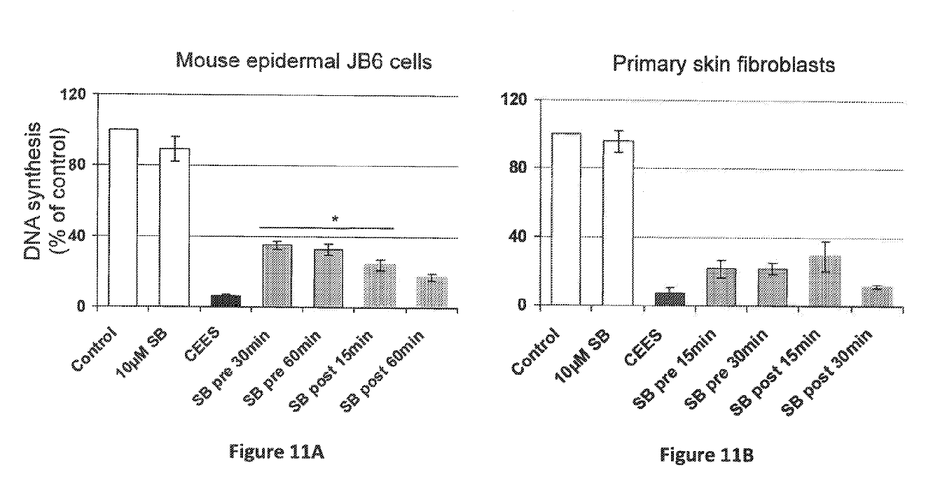

Cell proliferation was measured with the thymidine analog BrdU (5-bromo-2′-deoxyuridine) following its incorporation into newly synthesized DNA and its subsequent detection with an anti-BrdU antibody. Cells were cultured in vessels appropriate for microscopy, removed from the culture medium and labeled with a stock solution of BrdU by replacing the medium with BrdU labeling solution. After incubation, the labeling solution was removed and washed with PBS. The cells were then fixed, permeabilized, and acid-washed. Incorporated BrdU was detected by removing the solution and adding 1 mL of antibody staining buffer. The anti-BrdU primary antibody was added, and the cells were incubated, washed with Triton® X-100 permeabilization buffer, fluorescently labeled secondary antibody was added and the cells were again incubated before imaging. Silibinin treatment at 10 μM concentration 30 min and 60 minutes before and after CEES exposure for 24 h results in prevention or reversal of CEES-induced decrease in DNA synthesis in mouse epidermal JB6 cells, and primary skin fibroblasts (FIGS. 11A and 11B, respectively).

### C. Commet Assay for DNA Damage in the Skin Epidermal Cells and Fibroblasts

DNA damage was measured using single cell gel electrophoresis (SCGE) or alkaline comet assay (pH greater than or equal to 13) and staining with 3 mg/mL of PI. Briefly, cell suspension (300 mL) from the exposed and treatment groups after 1 h was mixed with 1% low-melting point agarose and was added to slides pre-coated with 1% normal-melting point agarose. These slides were then incubated O/N at 4 uC in lysis solution, washed, left for DNA unwinding and subjected to electrophoresis for 20 min at 22 V and 200 mA. Next, slides were neutralized in 500 mM Tris-HCl, pH 8.0, washed with H₂O and dried 0/N after staining with 3 mg/mL of PI. Fluorescence of the DNA in cells and in comets seen in case of DNA damage was scored using a Nikon invert microscope at 6200 magnification, and images were captured using an attached CoolSNAPES CCD camera. One hundred and fifty cells, 50 each on triplicate slides were captured and tail extent moment (TEM; product of tail length and percentage tail DNA) scored using Komet 5.5 software (ANDOR Technology).

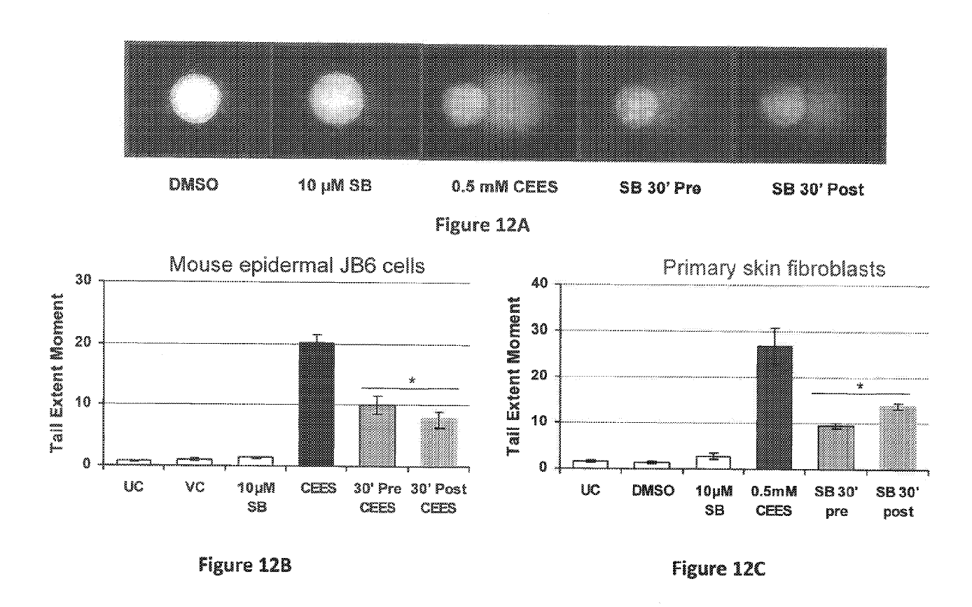

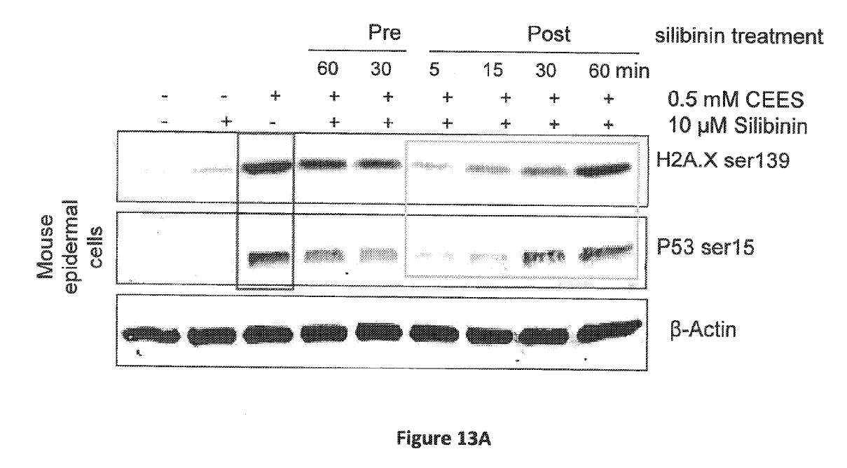

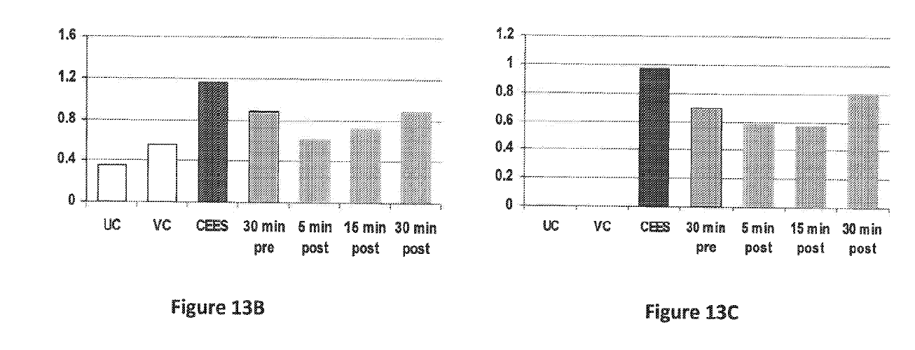

Cytotoxic effects of CEES are associated with its DNA damaging properties and the inventors have shown CEES-induced DNA damage in the form of increased TEM indicating damaged DNA in the cell (comet assay), H2A.X ser139 and p53 ser15 phosphorylation, in both JB6 cells and fibroblasts. Accordingly, the prevention and rescue efficacy of silibinin on CEES-induced DNA damage employing these established biomarkers resulted in the representative fluorescence micrographs of DNA comets (FIG. 12A) and their quantification, silibinin pre- and post-treatment caused a reduction or reversal in CEES-induced TEM in both JB6 cells (FIG. 12B), and fibroblasts (FIG. 12C). Similarly, as shown by immunoblots (FIG. 13A) and their quantifications (FIGS. 13B and 13C), silibinin also caused a reduction and reversal in CEES-induced H2A.X (FIG. 13B) and p53 phosphorylation (FIG. 13C).

Together, these findings demonstrate that the chemical properties of CEES are similar to that of sulfur mustard, and that CEES, like sulfur mustard, induces vesiculation and is an alkylating agent, although CEES possesses monofunctional alkylating properties and is therefore less toxic compared to sulfur mustard. Finally, these studies show that silibinin formulations of this disclosure possess strong antioxidant, anti-inflammatory, anti-angiogenic, anti-metastatic, and DNA repair properties that can both prevent and treat skin injuries from oxidative stress, inflammation, and activation of proteases produced by the vesicant models of skin injury investigated in this Example.

The foregoing examples have been presented for purposes of illustration and description. Furthermore, the description is not intended to limit this disclosure to the form disclosed herein. Consequently, variations and modifications commensurate with the above teachings, and the skill or knowledge of the relevant art, are within the scope of the present invention. The embodiments described hereinabove are further intended to explain the best mode known for practicing this disclosure and to enable others skilled in the art to utilize this disclosure in such, or other, embodiments and with various modifications required by the particular applications or uses of the present invention. It is intended that the appended claims be construed to include alternative embodiments to the extent permitted by the prior art.

1. A topical formulation comprising:
a. between about 0.1% (w/w) and about 10% (w/w), silibinin;
b. a physiologically acceptable carrier, comprising at least one of cetyl alcohol, sorbitol, glyceryl stearate, niacinamide, polysorbate 80, tocopherol, dimethicone, aloe barbadensis leaf juice, and sodium hyaluronate; and,
c. a final formulation pH between pH 4 and pH 6.5.

2. The topical formulation of claim 1, wherein the silibinin is present in concentrations between about 0.2% (w/w) and about 5% (w/w).

3. The topical formulation of claim 1, wherein the silibinin is present in a concentration of about 2% (w/w).

4. The topical formulation of claim 1, wherein the silibinin is present in a concentration of about 1% (w/w).

5. The topical formulation of claim 1, wherein the silibinin is predominately present as silibinin diastereomer A.

6. The topical formulation of claim 1, wherein the silibinin is predominately present as silibinin diastereomer B.

7. The topical formulation of claim 1, wherein the silibinin is present as a mixture of silibinin diastereomers A and B in a ratio of approximately 1:1.

8. The topical formulation of claim 1, wherein the formulation is substantially free of one or more lignans selected from silydianin, silychristin isosilybin, sauriol, licarin, saucernetin, saucerneol, niranthin, Phyllanthin, manassantins, matairesinol, hydroxymatairesinol, oxomatairesinol, saminol, americanin, arctiin, arctigenin, lariciresinol, isolariciresinol, secoisolariciresinol, secoisolariciresinol diglycoside, rubrisandrin, egonol, masutakeside, styraxlignolide, lappaol, diarctigenin, interiotherin, schisandrol, schisandrin, sesamin, sesaminol, episesamin, episesaminol, sesamolin, verbascoside, tetrahydrocurcumin, rosmarinic acid, chlorogenic acid, guaiaretic acid, dihydroguiaretic acid, nor-dihydroguiaretic acid, alpha-conidendrin, liovil, picearesinol, syringaresinol, and nortrachelogenin.

9. The topical formulation of claim 1, wherein the formulation is completely fee of one or more lignans selected from silydianin, silychristin isosilybin, sauriol, licarin, saucernetin, saucerneol, niranthin, Phyllanthin, manassantins, matairesinol, hydroxymatairesinol, oxomatairesinol, saminol, americanin, arctiin, arctigenin, lariciresinol, isolariciresinol, secoisolariciresinol, secoisolariciresinol diglycoside, rubrisandrin, egonol, masutakeside, styraxlignolide, lappaol, diarctigenin, interiotherin, schisandrol, schisandrin, sesamin, sesaminol, episesamin, episesaminol, sesamolin, verbascoside, tetrahydrocurcumin, rosmarinic acid, chlorogenic acid, guaiaretic acid, dihydroguiaretic acid, nor-dihydroguiaretic acid, alpha-conidendrin, liovil, picearesinol, syringaresinol, and nortrachelogenin.

10. The topical formulation of claim 1, further comprising at least one compound that enhances the penetration of silibinin into skin.

11. The formulation of claim 1, having a final formulation pH between pH 2 and pH 5.5.

12. The formulation of claim 1, having a final formulation pH between pH 4 and pH 5.0.

13. The formulation of claim 1, having a final formulation pH between pH 4.5 and pH 5.0.

14. The formulation of claim 1, having a final formulation pH of about pH 4.5.

15. The formulation of claim 1, having a final formulation pH of about pH 5.0.

16. The formulation of claim 1, having a final formulation pH of about pH 5.5.

17. The formulation of claim 1, having a final formulation pH of about pH 6.0.

18. The formulation of claim 1, having a final formulation pH of about pH 6.5.

19. The topical formulation of claim 1, further comprising at least one sunscreen selected from the group consisting of metallic oxides, zinc oxide, zirconium oxide, iron oxide, derivatives of dibenzoyl methane, cinnamic acid esters, diphenylacrylic acid esters, benzophenone, camphor, p-aminobenzoic acid esters, o-aminobenzoic acid esters, salicylic acid esters, benzimidazoles, symmetrically or unsymmetrically substituted 1,3,5-triazines, monomeric and oligomeric 4,4-diarylbutadienecarboxylic acid esters and -carboxylic acid amides, ketotricyclo(5.2.1.0)decane, benzalmalonic acid esters, 1-(4-tert.-butylphenyl)-3-(4′-methoxyphenyl)propane-1,3-dione (Parsol™ 1789), 1-phenyl-3-(4′-isopropylphenyl)-propane-1,3-dione, 3-(4′-methylbenzylidene)-D,L-camphor, 4-(dimethylamino)-benzoic acid 2-ethylhexyl ester, 4-(dimethylamino)benzoic acid 2-octyl ester, 4-(dimethylamino)-benzoic acid amyl ester, 4-methoxycinnamic acid 2-ethylhexyl ester, 4-methoxycinnamic acid propyl ester, 4-methoxycinnamic acid isopentyl ester, 2-cyano-3,3-phenylcinnamic acid 2-ethylhexyl ester (Octocrylene), salicylic acid 2-ethylhexyl ester, salicylic acid 4-isopropylbenzyl ester, salicylic acid homomethyl ester (3,3,5-trimethyl-cyclohexyl salicylate), 2-hydroxy-4-methoxybenzophenone, 2-hydroxy-4-methoxy-4′-methylbenzophenone, 2,2′-dihydroxy-4-methoxybenzophenone, 4-methoxybenzmalonic acid di-2-ethylhexyl ester, 2,4,6-trianilino-(p-carbo-2′-ethyl-1′-hexyloxy)-1,3,5-triazine, dimethicodiethylbenzal malonate, dioctyl butamido triazone, 2,4-bis-[5-1(di-methylpropyl)benzoxazol-2-yl-(4-phenyl)-imino]-6-(2-ethyl hexyl)-imino-1,3,5-triazine, 2-phenylbenzimidazole-5-sulfonic acid, phenylene-1,4-bis-(2-benzimidazyl)-3,3′-5,5′-tetrasulfonic acid, alkaline earth metals, ammonium, alkylammonium, alkanolammonium and glucammonium salts, sulfonic acid derivatives of benzophenone, sulfonic acid derivatives of 3-benzylidenecamphor, and mixtures of these sunscreen compounds.

20-30. (canceled)

31. A method of treating a skin condition comprising applying a therapeutically-effective amount of a topical formulation comprising between about 0.1% (w/w) and about 10% (w/w), silibinin; a physiologically acceptable carrier; and, a final formulation pH between pH 1 and pH 6.5, to a topical area of the skin, scalp, hair, lips or nails, of a subject in need of such treatment, wherein the skin condition is selected from the group consisting of:
a) oxidative damage to the skin, and photoaging of the skin caused by ultraviolet radiation;
b) skin inflammation, photocarcinogenesis (such as actinic keratosis/non-melanoma skin cancers), chemically-induced skin damages (such as contact dermatitis), irritant dermatitis, dermatitis/atopy, and facial redness/erythema associated with acne rosacea and skin aging;
c) skin wounds, environmentally-induced skin damage (such as UVA and UVB-induced skin damage including sunburn/tanning/skin thickening), chemically-induced skin damage caused by cytotoxic and vesicant chemical warfare agents (such as mustard pas, sulfur mustards, and nitrogen mustards), environmentally-induced skin damage (such as radiodermatitis/fibrosis associated with radiotherapy), and traumatic bruising/post-procedure bruising/senile purpura; and,
d) xerosis, dry skin, and dyspigmentation of the skin.

32-38. (canceled)
